(19) United States
(12) Patent Application Publication
(10) Pub. No.: US 2015/0362428 A1
(43) Pub. Date: Dec. 17, 2015

(54) SYSTEM AND METHOD FOR ELIMINATION OF FRESNEL REFLECTION BOUNDARY EFFECTS AND BEAM STEERING IN PULSED TERAHERTZ COMPUTED TOMOGRAPHY

(71) Applicants: John F. Federici, Westfield, NJ (US); Suman Mukherjee, East Newark, NJ (US)

(72) Inventors: John F. Federici, Westfield, NJ (US); Suman Mukherjee, East Newark, NJ (US)

(21) Appl. No.: 14/302,741

(22) Filed: Jun. 12, 2014

(51) Int. Cl. G01N 21/3581; G01N 33/36; G01N 33/44

(52) U.S. Cl. CPC G01N 21/3581; G01N 33/442; G01N 33/36

(57) ABSTRACT

A method of reducing boundary effect in a THz-CT image includes correcting steering of the THz beam and/or Fresnel reflection prior to reconstruction of the THz image, the method including tomographically scanning an object at selected rotational positions to obtain a plurality of projection slices, and prior to reconstructing the THz-CT image using a Radon transformation, applying to each of the plurality of projection slices an algorithm to determine a location of left and right edges of the object relative to the center of rotation of the object, determining a range of values for which the measured attenuation is not instrument limited based on a maximum detectable attenuation, and applying a further algorithm representing the corrected attenuation projection array data which will be inverted using the Radon transformation, the algorithm including an experimentally measured attenuation, correction for beam steering, and accounting for Fresnel reflection loss at the incident and exiting air-object interfaces.

### FIELD OF THE INVENTION

The present invention relates to the field of terahertz (THz) imaging, more specifically the area of non-destructive imaging and imaging for structural defect evaluation in the THz regime.

### BACKGROUND OF THE INVENTION

THz spectroscopy and imaging has received considerable attention for non-destructive evaluation of various materials including polymers, pharmaceuticals, detection of concealed weapons, and explosives. See, B. B. Hu and M. C. Nuss, Optics Letters. 20(16): p. 1716-1718 (1995); D. Mittleman, IEEE Journal of selected optics in quantum electronics, 2(3): p. 679-692 (1996); S. Wang and X-C Zhang, Journal of Physics D, 37(4): p. R1-R36 (2004); D. Mittleman et al., Optics Letters, 22(12): p. 904-906 (1997). The technique has also been applied to non-destructive evaluation of moisture content of many materials, including grain, leaves, wood, as well as polymers. See, A. Brahm et al., Infrared, Millimeter and Terahertz Waves (IRMMW-THz), 2011 36th International Conference: (2011); T. Buma and T. B. Norris, Applied Physics Letters, 84(12): p. 2196-2198 (2004); Wenfeng Sun et al., The European Conference on Lasers and Electro-Optics, Munich, Germany: (2011); Li Qi et al., Journal of Infrared, Millimeter, and Terahertz Waves, 33(5): p. 548-558 (2012); A. Wei-Min-Lee et al., Opt. Lett., 37(2): p. 217-219(2012); B. Ferguson et al., Optics Letters, 27(15): p. 1312-1314 (2002); M. Bessou et al., Appl. Opt., 51(28): p. 6738-6744 (2012); D. J. Roth at al., 69(9): Materials Evaluation, p. 1090-1098 (2011). THz spectroscopy and imaging has been discussed as a non-destructive evaluation (NDE) tool of natural cork enclosures. B. Recur et al., Optics Express, 19(6): p. 5105-5117 (2011). NDE of natural cork has been demonstrated by imaging the internal crack, voids, and grain structure of natural cork samples. Y. Hor, J. F. Federici, and R. L. Wample, Applied Optics, 2008. 47: p. 72-78.

THz Time-Domain Spectroscopy (THz-TDS) and imaging is a coherent measurement technology which is based on the measurement of a THz pulse in the time-domain. The Fourier transform of the pulse waveform gives measurement of both the frequency dependent phase as well as amplitude of the THz pulse. Time-domain THz waves provide temporal and spectroscopic information that enables development of various three-dimensional (3D) terahertz tomography imaging modalities. D. Mittleman, IEEE Journal of selected optics in quantum electronics, 2(3): p. 679-692 (1996); S. Wang and X-C Zhang, Journal of Physics D, 37(4): p. R1-R36 (2004). The interaction between a coherent THz pulse and an object provides rich information about the object under study; therefore, three-dimensional terahertz imaging is a very useful tool to inspect or characterize several types of objects.

X-ray computed tomography (CT) is an excellent methodology to measure the 3D cross sectional images of materials and much of the same methodology has been adapted in the THz and millimeter wave regions. For example, a variety of instrumentation hardware have been used for THz tomography systems including all-electronic 3D computed THz tomography operating in the 230-320 GHz frequency range, 3D THz imaging using single cycle THz pulses, continuous wave (CW) terahertz tomography with phase unwrapping, THz-CT using CW a gas laser operating at 2.25 THz, and THz optical coherence tomography based on quantum cascade lasers.

### SUMMARY OF THE INVENTION

The vastly different spatial scales of x-rays and THz radiation are such that the reconstruction methodologies which are routinely employed in the x-ray range may not be optimal in the THz range. X-rays, due to their nature, travel through the target material in almost straight lines without much refraction. Due to the refractive index change at material boundaries, THz waves are susceptible to refraction as well as loss of signal due to Fresnel reflection from boundaries. Due to the enhanced loss of THz power at a boundary due to both Fresnel reflection losses as well as refractive losses, reconstructed THz-CT images exhibit enhanced attenuation at the boundaries and also a distortion of the boundary shapes. Examples of the boundary effect can be seen in many instances concerning THz CT imaging. See, e.g., S. Wang and X-C Zhang, Journal of Physics D, 37(4): p. R1-R36 (2004); D. Mittleman et al., Optics Letters, 22(12): p. 904-906 (1997); B. Ferguson et al., Physics in Medicine and Biology, 47(21): p. 3735-3742 (2002); X-C Zhang, Philosophical Transactions of The Royal Society A, 362: p. 283-299 (2003); W. L. Chan et al., Reports on Progress in Physics, 70(8): p. 1325-1379 (2007). Only in the case of a low refractive index contrast material can the refractive and reflective effects be ignored in the image reconstruction. See, Li. Qi et al., Journal of Infrared, Millimeter, and Terahertz Waves, 33(5): p. 548-558 (2012); A. Wei-Min-Lee et al., Opt. Lett., 37(2): p. 217-219(2012); K. Hyeongmun et al., Optical Society of America (2012); S. A. Nishina et al., SAE International Journal of Fuels and Lubricants, 5(1): p. 343-351 (2012).

Other examples of adapting X-ray CT to the THz range include incorporating Gaussian beam properties in the image reconstruction process to improve the quality of the reconstructed images. See, B. Recur et al., Optics Express, 20(6): p. 5817-5829 (2012), in which Gaussian beam properties were simulated and when the beam properties were included, the reconstructed images showed differences compared to standard reconstructed images.

Some studies of boundary artifact phenomenon have included attempts to remove the prominent boundary effect phenomena by different methodologies. E. Abraham et al., Optics Communications, 283(10): p. 2050-2055 (2010) introduced a multi-peak averaging method to eliminate boundary effects due to refraction losses of the THz beam inside the material. As the THz beam propagates through the material, it suffers refraction inside the material and thus produces multiple peaks—instead of a single peak—when it passes through a material. In Abraham et al., a time delay from a particular peak was used to generate tomographic images. However, the presence of multiple peaks makes it difficult to choose the correct peak for time delay measurement. By averaging several of the peaks and considering the time delay of that averaged peak, the boundary effect can be reduced. In applying this technique to 3D THz-CT images of a Teflon cylinder (refractive index 1.37) with a hole formed in it, the effect of the boundaries is reduced, but the visibility of boundaries are still artificially enhanced in the reconstructed image.

For NDE of materials for which the internal structure is not uniform, artifacts in the reconstructed image can mask the subtle but important contrast in a sample's internal structure. In a previous study by the present inventors of internal defects in natural cork, pulsed THz-CT was used to reconstruct the cork's internal structure. S. Mukherjee and J. F. Federici, IRMMW-THz 2011—36th International Conference on Infrared, Millimeter, and Terahertz Waves, Houston, Tex., USA: p. 85 (2011). However, the strong boundary artifact made resolution of the mass density variations, cracks, voids, and channels of the cork structure difficult to discern. For natural cork, it is the internal structure which is thought to determine the gas diffusion properties of natural cork which are essential for the functionality of natural cork as barrier to gas and liquid diffusion. A. J. Teti et al., Infrared Milli. Terahz. Waves, 32: p. 513-527 (2011); J. F. Federici, IEEE Transactions on Terahertz Science and Technology, 33(2): p. 97-126 (2012).

Artificially large boundaries are produced in reconstructed THz-CT images due to the finite beam size of the probing THz radiation as well as strong refractive effects in the THz range including refractive power losses and beam steering. Of these effects, the beam steering is found to introduce the largest distortion in the THz projection data. Embodiments of the present invention provide correction algorithms for objects which can be applied to THz projection array data prior to reconstruction of the THz-CT image using Radon transformations. The presently disclosed subject matter invention corrects the edges of the projection data for the finite THz beam size as well as beam steering and Fresnel reflection losses. When algorithms in certain embodiments of the present invention are applied to plastic rods, for example, the artifically large attenuation near the boundary of the cylindrical sample is removed. Defects such as holes which are present near the central region of the sample are reproduced using correction algorithms of the present invention. When the defects are located near the periphery of the sample, the correction algorithms of certain embodiments of the present invention indicate the presence of the defect. When the algorithms are applied to a low refractive index (˜1.1) material such as natural cork, the boundary effect is significantly reduced in the reconstructed images enabling visualization of the cork's internal structure including holes and lenticels.

The present inventors have surprisingly found that THz CT can be an effective NDE tool if the general shape of an object under test is known. If the shape and refractive index of a ‘standardized’ object is known a-priori, then the effects of Fresnel reflection and refraction may be removed from CT projection data prior to applying an inverse Radon transformation to reconstruct the image. By removing the boundary artifacts, the discrimination of internal structure of a test object compared to the ideal ‘standardized’ object is greatly improved.

An example of one known shape for NDA is the shape of cylindrically shaped natural cork stoppers; the outer size and shape of each sample is essentially the same from sample to sample. Removing the boundary artifacts enables a more detailed reconstruction of each sample's internal structure which is so critical to the functionality of the stoppers with regard to their gas and liquid diffusion properties. Thus, a method is provided for removing boundary artifacts in THz CT imaging for cylindrically shaped objects.

In accordance with one or more embodiments a method of reducing boundary effect in a THz-CT image includes correcting steering of the THz beam and/or Fresnel reflection prior to reconstruction of the THz-CT image, the method including tomographically scanning an object at selected rotational positions to obtain a plurality of projection slices, and prior to reconstructing the THz-CT image, applying to each of the plurality of projection slices an algorithm to determine a location of left and right edges of the object relative to the center of rotation of the object, determining a range of values for which the measured attenuation is not instrument limited based on a maximum detectable attenuation, and applying a further algorithm representing the corrected attenuation projection array data, the algorithm including an experimentally measured attenuation, correction for beam steering, and accounting for Fresnel reflection loss at the incident and exiting air-object interfaces.

The method may include reconstructing the THz-CT image such as by using a Radon transformation.

In accordance with another embodiment a method of reducing boundary effect in a THz-CT image includes correcting steering of the THz beam and/or Fresnel reflection prior to reconstruction of the THz-CT image, the method including tomographically scanning an object at selected rotational positions to obtain a plurality of projection slices, and prior to reconstructing the THz-CT image, applying to each of the plurality of projection slices an algorithm

$${A}_{edge} ( {l}_{R} ) = - {log}_{e} [ \frac{1}{2} + \frac{1}{2} erf ( \frac{\sqrt{2} R}{{a}_{0}} ( {l}_{R} - 1 ) ) ]$$

to determine a location of left and right edges of the object relative to the center of rotation of the object, determining a range of l_{R} values for which the measured attenuation is not instrument limited based on a maximum detectable attenuation, applying an algorithm comprising the correction terms αLᵣ=ln(T(l_{R}))+ln(Tₛₜ(l_{R}))+ln(1−Rₚₐ)±ln(1−Rₐₚ) to the range of l_{R} values, wherein αLᵣ represents the corrected attenuation projection array data, −ln(T(l_{R})) is an experimentally measured attenuation, ln(Tₛₜ(l_{R})) is the correction for beam steering, and ln(1−Rₚₐ) and ln(1−Rₐₚ) account for Fresnel reflection loss at the incident and exiting air-object interfaces.

The method may further include applying an algorithm Aₜₕ(l_{R})=α₀L(l_{R})=α₀2R\sqrt{1−l_{R}²} where l_{R}=l/R and R is the radius of the object, to fill in projection array data as a function of l_{R} in a blind region from the object's edges to a boundary of corrected data.

The object may be cylindrical, and may be any material such as plastic, natural cork, etc. The object may be scanned horizontally and/or vertically at selected intervals, such as intervals in the range of 1 mm-5 mm.

The method may include reconstructing the THz-CT image such as by using a Radon transformation.

In another embodiment a non-transitory, computer readable storage medium containing a computer program is provided, which when executed by a computer processor causes the computer processor to perform actions, the actions including correcting steering of a THz beam and/or Fresnel reflection prior to reconstruction of a THz-CT image, including applying to each of a plurality of projection slices obtained by tomographically scanning an object at selected rotational positions an algorithm

$${A}_{edge} ( {l}_{R} ) = - {log}_{e} [ \frac{1}{2} + \frac{1}{2} erf ( \frac{\sqrt{2} R}{{a}_{0}} ( {l}_{R} - 1 ) ) ]$$

to determine a location of left and right edges of the object relative to the center of rotation of the object, determining a range of l_{R} values for which the measured attenuation is not instrument limited based on a maximum detectable attenuation, applying an algorithm comprising the correction terms αLᵣ=−ln(T(l_{R}))+ln(Tₛₜ(l_{R}))+ln(1−Rₚₐ)+ln(1−Rₐₚ) to the range of l_{R} values, wherein αLᵣ represents the corrected attenuation projection array data, −ln(T(l_{R})) is an experimentally measured attenuation, ln(Tₛₜ(l_{R})) is the correction for beam steering, and ln(1−Rₚₐ) and ln(1−Rₐₚ) account for Fresnel reflection loss at the incident and exiting air-object interfaces.

In one embodiment the object cylindrical, and may be any material such as plastic, natural cork, etc.

The non-transitory, computer readable storage medium containing a computer program may be operable to cause the computer processor to apply an algorithm Aₜₕ(l_{R})=α₀L(l_{R})=α₀2R\sqrt{1−l_{R}²} where l_{R}=1/R and R is the radius of the object, to fill in projection array data as a function of l_{R} in a blind region from the object's edges to a boundary of corrected data.

The non-transitory, computer readable storage medium containing a computer program may be operable to cause the computer processor to reconstruct the THz-CT image such as by using a Radon transformation.

In another embodiment, a apparatus includes a processor operating to perform actions in response to executing computer program instructions, the actions including correcting steering of a THz beam and/or Fresnel reflection prior to reconstruction of a THz-CT image, including applying to each of a plurality of projection slices obtained by tomographically scanning an object at selected rotational positions an algorithm

$${A}_{edge} ( {l}_{R} ) = - {log}_{e} [ \frac{1}{2} + \frac{1}{2} erf ( \frac{\sqrt{2} R}{{a}_{0}} ( {l}_{R} - 1 ) ) ]$$

to determine a location of left and right edges of the object relative to the center of rotation of the object, determining a range of l_{R} values for which the measured attenuation is not instrument limited based on a maximum detectable attenuation, applying an algorithm comprising the correction terms αLᵣ=−ln(T(l_{R}))+ln(Tₛₜ(l_{R}))+In(1−Rₚₐ)+ln(1−Rₐₚ) to the range of l_{R} values, wherein αLᵣ represents the corrected attenuation projection array data, ln(T(l_{R})) is an experimentally measured attenuation, ln(Tₛₜ(l_{R})) is the correction for beam steering, and ln(1−Rₚₐ) and ln(1−Rₐₚ) account for Fresnel reflection loss at the incident and exiting air-object interfaces. The object may be cylindrical, and may be a material such as but not limited to plastic, natural cork, etc.

The apparatus may be operable to apply an algorithm Aₜₕ(l_{R})=α₀L(l_{R})=α₀2R\sqrt{1−l_{R}²} where l_{R}=l/R and R is the radius of the object, to fill in projection array data as a function of l_{R} in a blind region from the object's edges to a boundary of corrected data.

The apparatus may be operable to cause the computer processor to reconstruct the THz-CT image using a Radon transformation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that those having ordinary skill in the art will have a better understanding of how to make and use the disclosed systems and methods, reference is made to the accompanying figures wherein:

FIG. 1(a) is a graphical depiction of a plot of experimentally measured average THz attenuation (solid) between 0.1-0.2 THz and theoretically calculated attenuation (dashed) of a solid, uniform Plexiglas rod in accordance with an embodiment of the presently disclosed subject matter;

FIG. 1(b) is a graphical depiction of a reconstructed pulsed THz-CT image of a horizontal slice through a uniform Plexiglas rod in accordance with an embodiment of the presently disclosed subject matter;

FIG. 2 is a schematic depiction of transmission of a THz beam with bending inside the material in accordance with an embodiment of the presently disclosed subject matter;

FIG. 3(a) is a schematic depiction of optimal propagation of THz radiation through collimating and focusing lenses in the absence of a sample and FIG. 3(b) is a schematic depiction of beam steering of THz radiation by a sample as an optical component in a system (Tx and Rx are abrreviations of transmitter and receiver, respectively) in accordance with an embodiment of the presently disclosed subject matter;

FIG. 3(c) is a block diagram of a computing system suitable for carrying out methods in accordance with one or more embodiments of the present invention;

FIG. 4 is a graphical depiction of predicted attenuation from ray-tracing simulation data (dotted grey curve) and quadratic fit (solid black curve overlaying gray dotted curve) in accordance with an embodiment of the presently disclosed subject matter;

FIG. 5 is a schematic depiction of a cylindrical object approaching a Gaussian beam which is propagating perpendicular to the page in accordance with an embodiment of the presently disclosed subject matter;

FIG. 6(a) is a graphical depiction of experimentally measured attenuation (solid line), ideal attenuation projection (gray dotted line) assuming no refraction, beam steering correction (dotted line) and predicted attenuation due to the edge of the sample blocking the THz beam (dashed line) in accordance with an embodiment of the presently disclosed subject matter;

FIG. 6(b) is a graphical depiction of corrected attenuation from Eq. (7) using the beam steering correction term determined from ray-tracing and Eq. (10) to determine the edges of the sample in accordance with an embodiment of the presently disclosed subject matter;

FIG. 7(a) is a graphical representation of a reconstructed pulsed THz-CT image of a circular cross-section through a homogeneous cylindrical Plexiglas rod after a correction algorithm is applied to the measured projection data in accordance with an embodiment of the presently disclosed subject matter;

FIG. 7(b) is a graphical representation of a plot profile through a diameter of the subject matter in FIG. 7(a).

FIG. 8(a) is a graphical depiction of a reconstructed 2D tomographic image of a plastic rod with a 1 mm hole near the centre without a correction algorithm applied;

FIG. 8(b) is a graphical depiction of a reconstructed 2D tomographic image of a plastic rod with a 1 mm hole near the centre with a correction algorithm applied in accordance with an embodiment of the presently disclosed subject matter;

FIG. 8(c) is a graphical depiction of a plot profile corresponding to the image of FIG. 8(a) taken through the diameter with no correction algorithm applied;

FIG. 8(d) is a graphical depiction of a plot profile corresponding to the image of FIG. 8(b) taken through the diameter with a correction algorithm applied in accordance with an embodiment of the presently disclosed subject matter;

FIG. 9(a) is a graphical depiction of a reconstructed 2D tomographic image of a plastic rod with a 5 mm hole near the center without a correction algorithm applied;

FIG. 9(b) is a graphical depiction of a reconstructed 2D tomographic image of a plastic rod with a 5 mm hole near the center with a correction algorithm applied in accordance with an embodiment of the presently disclosed subject matter;

FIG. 9(c) is a graphical depiction of a plot profile of the image of FIG. 9(a) taken through the diameter with no correction algorithm applied;

FIG. 9(d) is a graphical depiction of a plot profile of the image of FIG. 9(b) taken through the diameter with a correction algorithm applied in accordance with an embodiment of the presently disclosed subject matter;

FIG. 10(a) is a graphical depiction of a reconstructed 2D tomographic image of a plastic rod with a 5 mm hole near the periphery of the sample without a correction algorithm applied;

FIG. 10(b) is a graphical depiction of a reconstructed 2D tomographic image of a plastic rod with a 5 mm hole near the periphery of the sample with a correction algorithm applied in accordance with an embodiment of the presently disclosed subject matter;

FIG. 10(c) is a graphical depiction of a plot profile of the image of FIG. 10(a) taken parallel to the bottom of the page through the diameter with no correction applied;

FIG. 10(d) is a graphical depiction of a plot profile of the image of FIG. 10(b) taken parallel to the bottom of the page through the diameter with a correction algorithm applied in accordance with an embodiment of the presently disclosed subject matter;

FIG. 11(a) is a graphical depiction of a reconstructed 2D tomographic image of natural cork without a correction algorithm applied;

FIG. 11(b) is a graphical depiction of a reconstructed 2D tomographic image of natural cork with a correction algorithm applied in accordance with an embodiment of the presently disclosed subject matter;

FIG. 11(c) is a graphical depiction of a plot profile of the image of FIG. 11(a) taken parallel to the bottom of the page through the diameter with no correction applied; and

FIG. 11(d) is a graphical depiction of a plot profile of the image of FIG. 11(b) taken parallel to the bottom of the page through the diameter with a correction algorithm applied in accordance with an embodiment of the presently disclosed subject matter.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

In certain embodiments of the present invention boundary effect resulting from THz CT is minimized and/or effectively eliminated by correcting for the two most dominant phenomena, i.e., steering of the THz beam and Fresnel reflection, prior to image reconstruction. In some embodiments algorithms are provided for correcting the edges of the projection data for the finite THz beam size. Determining the shape and composition of an object prior to image reconstruction and applying the corrections described herein solves the problems of reflection artifacts associated with THz CT.

Thus, the presently disclosed methods permit THz CT to be an effective NDE tool for objects that ordinarily would not be amenable to effective THz CT due to a lack of precision. The presently disclosed methods take into account the shape and refractive index of the object to correct for refraction artifacts.

In the present invention, a standardized object is utilized to develop a baseline. This standardized object has a known shape and refractive index. This allows for the removal of the effects of Fresnel reflection and refraction from CT projection data prior to applying an inverse Radon transformation to reconstruct the image. These standardized objects may then be compared to test objects.

An example of one known shape for NDE is the shape of cylindrically-shaped natural cork stoppers, the outer size and shape of each sample is essentially the same from sample to sample. Removing the boundary artifacts enables a more detailed reconstruction of each sample's internal structure which is so critical to the functionality of the stoppers with regards to their gas and liquid diffusion properties. Thus, a method is provided for removing boundary artifacts in THz CT imaging for cylindrically shaped objects.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

For example, while the examples and experiments employ cork material and Plexiglass rods, those skilled in the art will recognize other materials may be employed in accordance with the teachings herein and the methods disclosed herein may be applied to other such materials.

### EXAMPLES AND EXPERIMENTS

The non-homogeneous internal structure of cork and significant sample-to-sample variations precludes natural cork as a model material for the development of correction algorithms. Therefore, in order to study the boundary effect and develop algorithms for removing boundary effect from THz-CT reconstructed images, several cylindrically-shaped plastic Plexiglas rods (real refractive index 1.54) were used as target material for algorithm development. Cylindrically shaped plastic rods provide a uniform, homogeneous material for development of boundary correction algorithms.

Four identical plastic rods were chosen for creating correction algorithms for certain embodiments of the present invention. Of these four rods, one rod was kept intact. In two of the samples, a uniform cylindrical-shaped hole (1 mm and 5 mm diameters respectively) was drilled near the geometric center of the cylinder. In the fourth sample, a cylindrically-shaped hole (5 mm diameter) was drilled near the periphery of the sample. These sample plastic rods were subjected to transmission scans by a pulsed THz beam.

The Plexiglas rod and natural cork samples were scanned in transmission using a Picometrix T-Ray 2000 system. Details of the experimental set-up have been discussed in the previous work of the inventors. See, e.g., S. Mukherjee and J. F. Federici, IRMMW-THz 2011—36th International Conference on Infrared, Millimeter, and Terahertz Waves, Houston, Tex., USA: p. 85 (2011), incorporated by reference herein in its entirety.

For tomographic scanning, samples were attached to a rotation stage such that the geometric center of the cylinder was nominally collinear with the rotation axis of the stage. Each sample was rotated in 2 degree intervals from zero to 360 degrees (up to 180 rotations) relative to its original position. Full tomographic scanning was obtained by vertically (resolution 1 mm) and horizontally (0.5 mm resolution) scanning a sample at each rotational position.

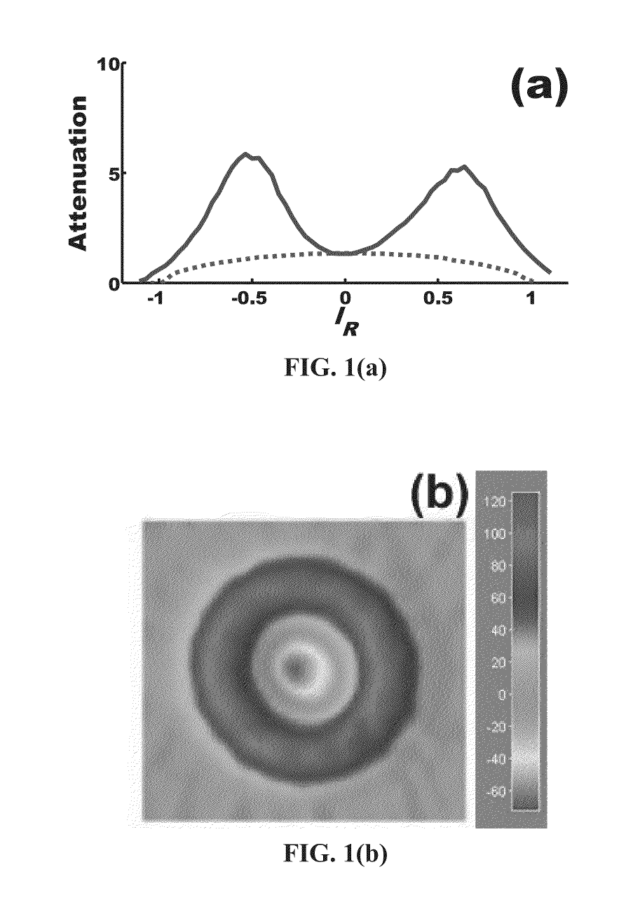

From the THz time-domain spectra, the average attenuation (A=ln(1/T)) was calculated, where T is the transmission at 0.15 THz for each scan position. For each horizontal slice through circular cross-section of the cylinder at a fixed rotational angle, the array of attenuation data represents a projection through the sample. See, S. Wang and X-C Zhang, Journal of Physics D, 37(4): p. R1-R36 (2004), incorporated by reference herein in its entirety. A typical projection for a solid Plexiglas rod is shown in FIG. 1(a). By measuring the projection array at each rotation angle, a 2D reconstruction of the slice was generated, using a MATLAB® built-in function for Radon transformation with filtered-back propagation. MATLAB® is a high-level language and interactive environment for numerical computation, visualization, and programming available from MathWorks of Natick, Mass. A typical reconstructed slice using the measured (uncorrected) projection arrays is shown in FIG. 1(b). A complete 3D reconstruction is achieved by stacking the reconstructed slices at each vertical position.

FIG. 1(a) shows a typical plot of the experimentally measured and ideal attenuation projection through a solid Plexiglas rod measured at 0.15 THz. The ideal projection data is calculated using measured values for the real refractive index and the attenuation coefficient of a homogenous sample. These values are measured by propagating the THz beam through the diameter of the sample and analyzing the resulting frequency dependent phase and magnitude.

The ideal projection assumes that the THz beam propagates straight through the sample. For this ideal projection, the attenuation should depend only on the material's attenuation coefficient α₀ and the path length through the sample. The path through the sample depends on the offset distance l relative to the geometric center of the circular cross-section

Aₜₕ(l_{R})=α₀L(l_{R})=α₀2R\sqrt{1−l_{R}²} (1)

where l_{R}=l/R, and R is the radius of the sample. According to Eq. (1), the center position corresponding to l_{R}=0 should exhibit the maximum attenuation with decreasing attenuation as the offset parameter increases away from the center diameter. However, in reality, the measured attenuation (FIG. 1(a)) is a minimum at the center position and increases with increasing |l_{R}|. Consequently, the reconstructed 2D tomographic image of a horizontal slice through the rod shows an enhanced attenuation at the boundaries of the rod (FIG. 1(b)). Ideally, the reconstructed attenuation coefficient should be uniform throughout the material.

In embodiments of the present invention an algorithm is provided that eliminates the effect of the Fresnel reflection and beam steering, so that the anomalous attenuation shown in the reconstructed image (FIG. 1(b)) is removed. This is done by correcting for the effects of Fresnel reflection and beam steering from attenuation projection data prior to reconstructing the image using Radon transformations.

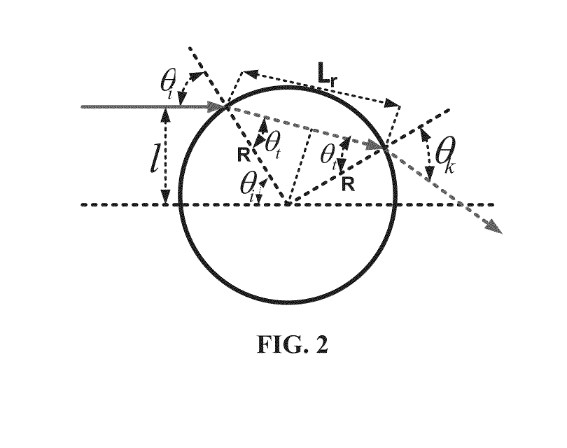

Now referring to FIG. 2 a schematic depiction is provided of a THz beam transmitting through a circular cross section of a cylindrical rod. From FIG. 2, the distance that the THz beam travels inside the material upon refraction clearly is not the same as if the beam were to travel in a straight line. Using the geometry of FIG. 2 with Snell's law of refraction nₐ sin θᵢ=nₚ sin θₜ, where θᵢ and θᵢ are the angles of the incident and refracted rays, it is straightforward to show that the path length which the THz beam travels inside of the sample upon refraction is Lᵣ=2R\sqrt{1−nₐ²l_{R}²/nₚ²}, where nₐ and nₚ are refractive index of air and plastic, respectively. In order to estimate the relative change in length, consider the refractive indices of the plexiglass (1.54) and air (1.0). If the ray were to travel at a distance l_{R}=½ from the centre axis, then the path length through the sample assuming that there is no refraction would be L=1.73R, while the path length with refraction would be Lᵣ=1.89R corresponding to a ˜9% increase in path length due to refraction.

In calculating the algorithms in certain embodiments of the present invention, the polarization of the incident THz wave is parallel to the plane of incidence. The Fresnel reflection losses are included using the power reflection coefficient

$$R = {( \frac{{n}_{t} cos {θ}_{i} - {n}_{i} cos {θ}_{t}}{{n}_{i} cos {θ}_{t} + {n}_{t} cos {θ}_{i}} )}^{2} \qquad ( 2 )$$

(Eugene Hecht, Optics, 4^{th} edn, (Addison Wesley, 2001), incorporated by reference herein in its entirety), where nᵢ, nₜ, θᵢ and θₜ are refractive index of the incident medium, refractive index of the transmitted medium, incidence and transmitted angle, respectively. Applying both Snell's law and Eq. (2) to both the air-plastic and plastic-air interfaces gives the following reflection losses at the two boundaries

$$\begin{matrix}{R}_{ap} ( {l}_{R} ) = {( \frac{{n}_{p} \sqrt{1 - {l}_{R}^{2}} - {n}_{a} \sqrt{1 - {n}_{a}^{2} {l}_{R}^{2} / {n}_{p}^{2}}}{{n}_{a} \sqrt{1 - {n}_{a}^{2} {l}_{R}^{2} / {n}_{p}^{2}} + {n}_{p} \sqrt{1 - {l}_{R}^{2}}} )}^{2} & ( 3 ) \\ {R}_{pa} ( {l}_{R} ) = {( \frac{{n}_{a} \sqrt{1 - {l}_{R}^{2} {n}_{a}^{2} / {n}_{p}^{2}} - {n}_{p} \sqrt{1 - {l}_{R}^{2}}}{{n}_{p} \sqrt{1 - {l}_{R}^{2}} + {n}_{a} \sqrt{1 - {l}_{R}^{2} {n}_{a}^{2} / {n}_{p}^{2}}} )}^{2} & ( 4 )\end{matrix}$$

The total transmission of a THz beam through the sample can be written as:

T(l_{R})=(1−Rₐₚ) exp(−αLᵣ)(1−Rₚₐ) (5)

where α is the power attenuation coefficient for the plastic. The first term of Eq. (5) represents the power loss when the THz beam refracts as it enters the sample. The second term represents the attenuation loss of the THz beam propagating through the sample, while the third term represents the reflective power loss as the beam exits the sample. It should be noted that this loss term can be up to 100% if the THz beam is totally internally reflected at the plastic-air interface.

As an estimate of the Frensel power losses for certain embodiments of the present invention, consider a beam travelling through the material at a distance l_{R}=½. Using the measured linear attenuation coefficient α=0.0311/mm and radius of the plastic rod (14 mm), the transmission of the THz beam through the material is estimated from Eq. (5) to be 0.41 including Fresnel reflection losses and roughly 0.47 when the losses are neglected. Therefore, inclusion of the Fresnel losses reduces the transmission by 14% which corresponds to an attenuation change of about 0.14.

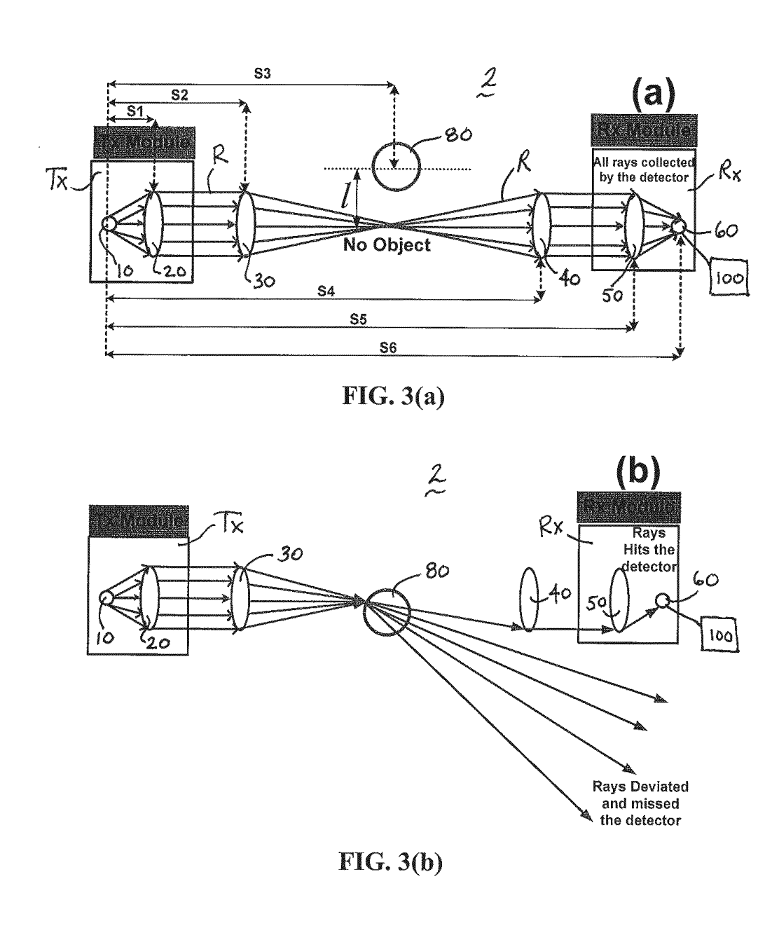

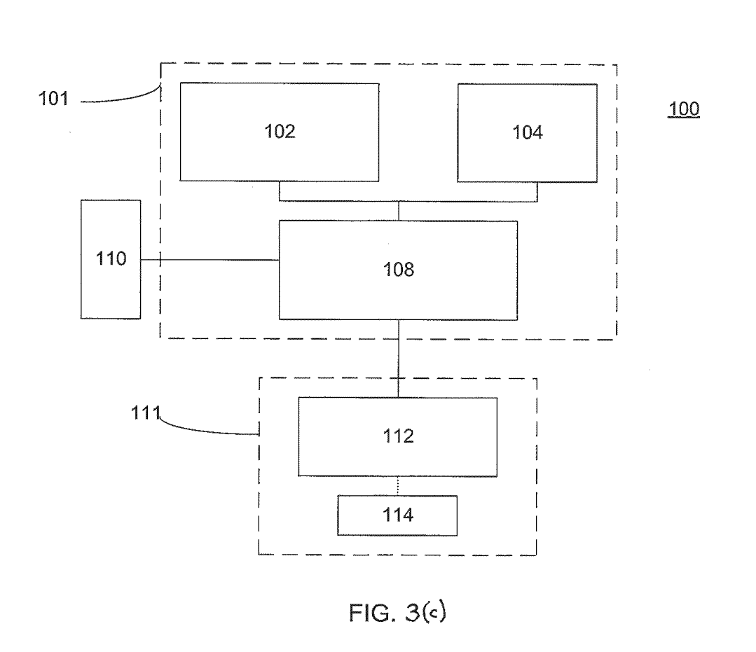

While the increase in sample path length and Fresnel losses increase the attenuation from what one would ideally expect for an undeviated beam, the magnitude of these losses is too small to explain the rapid increase in attenuation observed (in FIG. 1(a)) as the offset parameter l_{R} (in FIG. 2) of the THz beam relative to the geometric center of the cylindrical rod is increased on its own. In the absence of the sample, the lenses and other optical components of the THz system are aligned to optimize the THz power from the THz transmitter to the detector. The presence of any sample in the beam path becomes part of the optical system since refraction of the THz beam by the sample will steer the THz beam from its optimal path to the detector. Now referring to FIG. 3(b), when the cylindrical sample is off-center in the THz optical system, the resulting beam steering will reduce the measured THz transmission. Embodiments of the present invention correct for this beam steering phenomenon in addition to Fresnel losses as discussed above.

In order to predict the effects of beam steering on the THz-CT system, a ray-tracing software program BEAM4 (Stellar Software, Berkeley, Calif.) was used to model the optical system. A model system 2 may include a source 10, first lens 20, second lens 30, third lens 40, fourth lens 50 and detector 60. A transmission module Tx may include a source 10 and first lens 20, and a receving module Rx may include a fourth lens 50 and detector 60. Object 80 may be positioned between the transmission module Tx and a receving module Rx. A table of the optical components, their spatial locations, and physical sizes which were used for the ray-tracing calculation are listed in Table 1 and illustrated in FIGS. 3(a) and 3(b). Using the geometric ray tracing software, a fan of rays R was defined emerging from the source 10 and their progression traced through the optical components 20, 30, 40 and 50. The fan of rays simulates the THz beam as it propagates through the optical system. By counting the percentage of the rays which propagate through the optical system to the detector 60 as a function of the offset parameter l_{R}, the transmission Tₛₜ of THz radiation was estimated. It was assumed that when l_{R}=0, corresponding to optimal optical alignment, the transmission is a maximum (100%).

TABLE 1
Optical components, their locations and physical size
used in the experiments.

| | Distance from the | Diameter | Refractive | Curvature of |
|---|---|---|---|---|
| Components | source (mm) | (mm) | Index | the Surface |
| Source | 0 | 0.1 | | |
| First Lens | S1 = 76.2 | 38.5 | 1.5 | 0.05194/mm |
| Second Lens | S2 = 215.1 | 38.5 | 1.5 | 0.05194/mm |
| Scanned Object | S3 = 292.1 | 28 | 1.1 | 0.07142/mm |
| Third Lens | S4 = 368.3 | 38.5 | 1.5 | 0.05194/mm |
| Forth Lens | S5 = 571.5 | 38.5 | 1.5 | 0.05194/mm |
| Detector | S6 = 647.7 | 0.1 | | |

In one embodiment a system 2 may be operably connected to a computing system 100.

As will be apparent to the skilled artisan, one or more embodiments of the presently disclosed subject matter may include a computer or server coupled to one or more user computers over a network, such as the Internet. The computer and/or server and user computers are operable to carry out computing activity (e.g., the execution of suitable software code) in connection with implementing the functions and actions of systems disclosed and described herein.

By way of example, the server and/or the user computers may be implemented using known hardware, firmware, and/or software, as well as specialized software for carrying out specific functions and actions desirable for implementing embodiments of the invention. For example, with reference to FIG. 3(c), a system 100 may include a computer 101, which includes a data processing unit (or processor) 102 and a memory 104 operatively coupled by way of a data and/or instruction bus 106. The processor 102 may be implemented utilizing any of the known hardware, such as a digital microprocessor, a computer (such as a portable, a stationary and/or a distributed computing system), or any of the other known and/or hereinafter developed data processing units. The memory 104 may be implemented by way of separate hardware or may be disposed within the data processing unit 102, and any of the known hardware and/or software for implementing the memory function may be employed.

Data are preferably input to, and output from, the data processing unit 102 by way of an input/output device (or I/O interface) 108. Operators of the system 100 may desire to input software programs and/or data into the computer 101 by way of an external memory 110 that is coupled to the I/O interface 108 by way of a suitable link (such as a cable, wireless link, etc.) The external memory 110 may be implemented via a flash-drive, disc, remotely located memory device, etc.

The server and/or the user computers may also include an interface device 111, which is operatively coupled to the I/O interface 108 of the computer 101 via a suitable link, such as a cable, wireless link, etc. The interface device 111 includes at least one display 112, as well as an input device 114, such as a keyboard, mouse, voice recognition system, etc. The operators of the system 100, such as an IT professional or a researcher, preferably utilizes the interface device 111 to provide information to the computer 101 in connection with entering appropriate data and/or programs into the system 100.

The computer 101 manipulates data via suitable software code in accordance with various embodiments of the invention and may display results on the display 112 for consideration by the various operators (IT professionals, users, etc.). In accordance with well-known techniques, the results may also be stored within the memory 104 of the computer 101, output and saved on the external memory device 110, and/or provided in any of a number of other ways.

It is noted that the functional blocks illustrated in FIG. 3(c) may be partitioned as shown or may be partitioned in any other way, such as in an integral fashion. By way of example, the system 100 may be implemented utilizing a portable, stationary, or distributed computer operating under one or more suitable computer programs. Further, one or more of the functional blocks of the system 100 may be remotely located from the others, such as in a distributed (e.g., networked) system.

Irrespective of how the system 100 is implemented and/or partitioned, it preferably carries out one or more methods described herein.

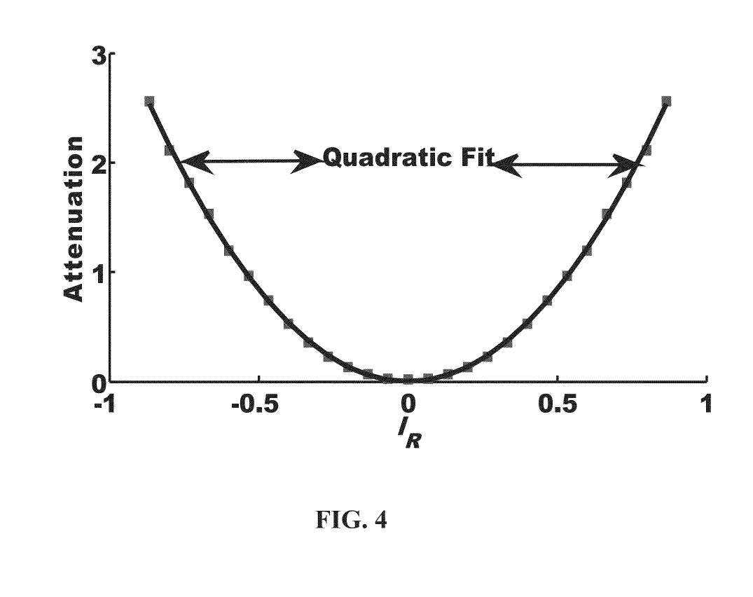

The simulated attenuation used in certain embodiments of the present invention due to beam steering Aₛₜ(l_{R})=ln(Tₛₜ(l_{R})) is shown in FIG. 4. In FIG. 4, a graphical depiction of predicted attenuation from ray-tracing simulation data (dotted curve) and quadratic fit (solid black curve overlaying dotted curve) in accordance with an embodiment of the presently disclosed subject matter is determined by −ln(Tₛₜ(l_{R}))=al_{R}²+bl_{R}+c where a is 2.94. The values for b and c are on the order of 10⁻¹⁶ and can be regarded as zero respectively. For |l_{R}|>1 (2R=28 mm), the attenuation is zero since the sample is not in the path of the THz beam. Note that as |l_{R}| increases above zero, the predicted attenuation due to beam steering Aₛₜ(l_{R}) continues to increase monotonically. In theory, the attenuation due to steering becomes infinite for large values of |l_{R}| due to extreme beam deviations as well as total-internal reflection of the THz beam at the sample-air exiting interface. In practice, the maximum correctable attenuation in the present invention is capped at roughly 4.6 because this is the typical maximum attenuation which is measured due to the signal-to-noise limitations of current THz spectroscopy systems. This limit is instrument specific and it could be any arbitrary number, more or less than 4.6 and it could vary depending upon the instrument used.

In order to define a correction term due to beam steering for certain embodiments of the present invention, the simulated attenuation is fitted to a quadratic polynomial given by ln(Tₛₜ(l_{R}))=al_{R}²+bl_{R}+c where a, b, and c are constants determined by the best fit to the data of FIG. 4. As will be apparent to the skilled artisan, the fitting parameters will change depending on the refractive index of the sample. Fitting of the data from FIG. 4 to higher order polynomials (4^{th} order, 6^{th} order and 8^{th} order polynomials) exhibits an improved fit as indicated by lower χ² values. But when the higher order polynomial fits are used as part of the correction algorithm, the corrected data for a homogenous, uniform plastic rod does not follow the ideal curve of Eq.(1). On the contrary, a quadratic ray-tracing correct term accurately reproduces Eq.(1). For this reason a quadratic polynomial has been used to fit the ray-tracing correction curve for certain embodiments of the present invention.

The effects of beam steering are included into Eq. (5). Using an additional factor Tₛₜ(l_{R}) which accounts for the effective transmission of the THz-CT system in the presence of beam steering

T(l_{R})=Tₛₜ(l_{R})exp(−αLᵣ)(1−Rₚₐ)(1−Rₐₚ) (6)

where the left-hand side of Eq. (6) is the measured THz transmission. Solving this equation for the parameter of interest αLᵣ gives

αLᵣ=ln(T(l_{R}))+ln(Tₛₜ(l_{R}))+ln(1−Rₚₐ)±ln(1−Rₐₚ) (7)

For certain embodiments of the present invention equation (7) illustrates the three correction terms which are applied to the measured THz attenuation prior to reconstructing the THz-CT image using a Radon transformation. The αLᵣ term represents the corrected attenuation projection array data which will be inverted using the Radon transformation. The −ln(T(l_{R})) term is the experimentally measured attenuation. The ln(Tₛₜ(l_{R})) term is the correction for beam steering, while the ln(1−Rₚₐ) and ln(1−Rₐₚ) terms account for the Fresnel's reflection loss at the incident and exiting air-plastic interfaces.

The shape of the measured attenuation near the sample boundaries results from the finite beam size of the probing THz beam. For large values of |l_{R}|>1.11, the sample is not in the THz beam path and the measured THz transmission (attenuation) is unity (zero). As the sample is scanned horizontally in l_{R}, the edge of the sample partially blocks the THz beam leading to an increase in measured attenuation. Due to the large angle of incidence near the edges of the sample, the beam steering is so severe as none of the light which enters the sample is able to propagate to the detector. The attenuation near the edges of the sample, therefore, are modelled assuming that the edge of the sample partially blocks the THz beam. Assuming that the THz beam propagates in the z direction as a Gaussian beam with a spot size of a₀ at the focus, the local intensity of the THz beam is given by

I(x,y)=I₀ exp(−2(x²+y²)/a₀²) (8)

(E. D. Hirleman, W. H. Stevenson, Applied Optics, 17(21): (1978), incorporated by reference herein in its entirety), where

$${I}_{0} = \frac{2 {P}_{0}}{π {a}_{0}^{2}}$$

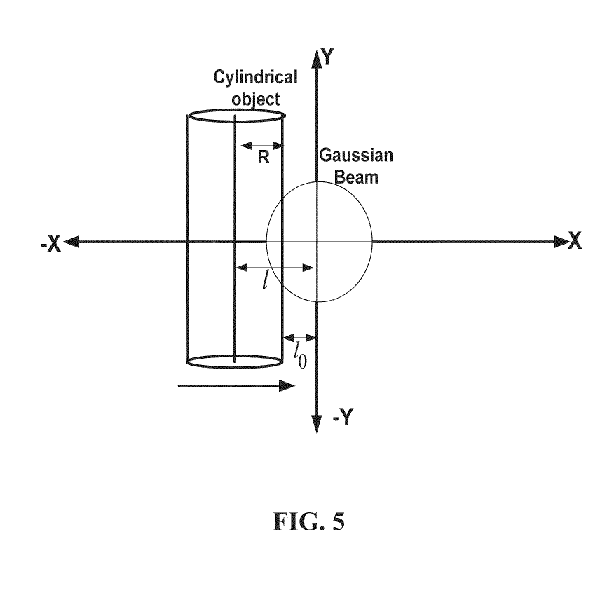

and P₀ is the total power of the beam at any cross section. The total power of THz radiation which gets blocked by the sample edge is calculated by integrating Eq. (8) over the transverse directions as illustrated in FIG. 5.

The measured transmittance near the sample's right edge (corresponding to positive values of l_{R}) is

$${T}_{edge} ( {l}_{R} ) = \sqrt{\frac{2}{π}} \frac{1}{{a}_{0}} {\int }_{- R ( {l}_{R} - 1 )}^{∞} exp ( - 2 {x}^{2} / {a}_{0}^{2} ) d x \qquad ( 9 )$$

where l_{R} represents the horizontal (ie. x) location of the sample's edge from the center of the beam. After integrating Eq. (9) and taking the negative of natural log on the right side of the expression, one derives the attenuation near the sample right edge as,

$${A}_{edge} ( {l}_{R} ) = - {log}_{e} [ \frac{1}{2} + \frac{1}{2} erf ( \frac{\sqrt{2} R}{{a}_{0}} ( {l}_{R} - 1 ) ) ] . \qquad ( 10 )$$

For the left edge (negative values of l_{R}), the equation is the same except the limits of integration range from negative infinity to −R(l_{R}+1).

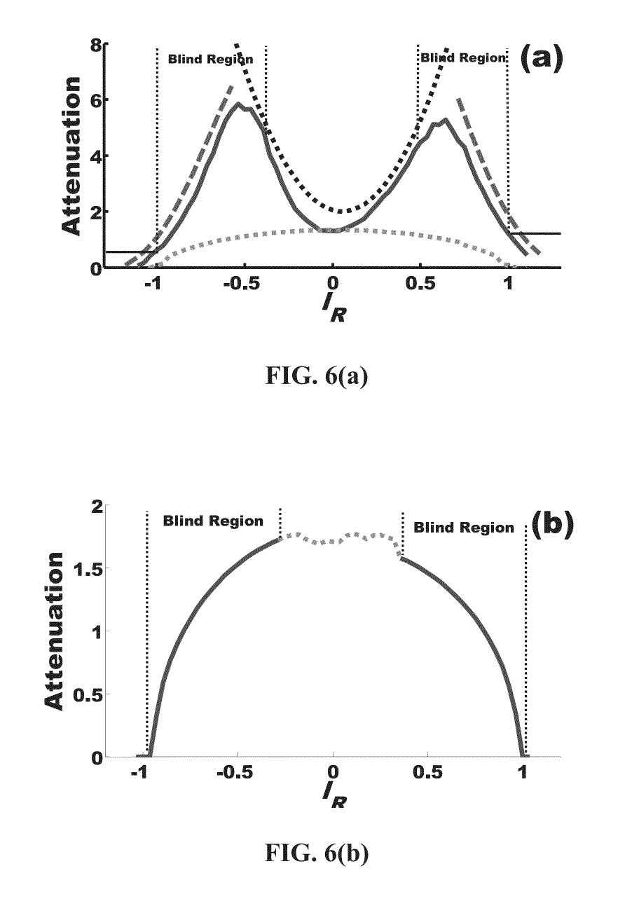

FIG. 6(a) shows a comparison of the measured THz attenuation −In[T (l_{R})] with beam steering correction term −ln[Tₛₜ(l_{R})]. The correction term has been offset vertically in the plot in order to compare the shapes of the two curves. Note that the similarity in the two curves strongly suggests that the large increase in attenuation with increasing offset parameter l_{R} is primarily due to beam steering. FIG. 6(a) is a typical plot of Eq. (10) for the left and right edges. From a best fit to the experimental data, the fixed THz beam spot size (a₀=2 mm) was extracted as well as the values of l_{R} for the left and right edges of the sample. The l_{R} position of the sample's physical edge is determined when the measured value for the attenuation A_{edge}(l_{R})=ln(2) corresponding to half of the THz beam being blocked by the sample's edge.

As part of the correction algorithm, one must account for the fact that the rotational axis for the tomography scans does not exactly coincide with the geometric axis of the cylindrical rod. Consequently, the first step in the correction algorithm for certain embodiments of the present invention is to use Eq. (10) to determine the left and right boundaries of the rod. Once these boundaries are determined, the offset between the rotational and geometric axes is used to apply correction terms of Eq. (6) for both Fresnel reflection losses and beam steering.

While the beam steering and Fresnel correction factors of Eq. (6) are used to correct the middle portion of the projection data for certain embodiments of the present invention, the THz-TDS system's detection limit as well as the severe beam steering when the THz beam passes near the edges of the rod implies that the measured attenuation for |l_{R}|>0.36 in FIG. 6(a) is an artifact and not related to the attenuation of the sample material. Near the edge of the sample, the deviation of the THz beam is so large that much of the beam from that region does not reach the detector. Since THz radiation which passes through this region is not detected, tomographic information from this region cannot be determined and thus that area is referred to as a “blind” region. In order to correct the attenuation projection data near the blind region of the sample, one must assume a functional form for the data in this range and match the experimental data to the measured values in the regions which are valid. Since the cylindrical rods are supposed to be homogenous and uniform, the expected functional form for the attenuation project array should ideally (in the absence of refraction) be due only to the attenuation coefficient α₀ of the material and the length of material through which the THz beam propagates.

At the physical edge corresponding to l_{R}=1, the attenuation through the material should have a limiting value of zero. Since the radius of the rod is known, the only free parameter in Eq. (1) is the attenuation coefficient α₀. This value is determined by matching the attenuation value from Eq. (1) to the corrected value of the measured attenuation from Eq. (7) at a fixed value of l_{R} corresponding to the attenuation detection limit of the THz-TDS system. As an example, FIG. 6(b) shows the corrected attenuation projection data for a fixed rotation angle and illustrates regions of experimentally corrected attenuation as well as regions near the periphery of the sample for which the attenuation is assumed to follow Eq. (1).

In summary, a correction algorithm of certain embodiments of the present invention may be applied as follows:

For each projection slice, use Eq. (10) to determine the location of the left and right edges of the cylinder relative to the center of rotation of the sample. Based on the maximum detectable attenuation (about ˜4.6 for the instrument used in this test), determine the range of l_{R} values for which the measured attenuation is not instrument limited. Apply the correction terms of Eq. (7) to that range of l_{R} parameters. Use the ideal form for the attenuation Eq. (1) to fill in projection array data as a function of l_{R} in the “blind” region from the sample's edges to the boundary of the valid corrected data. Repeat for each projection slice for all rotation angles of the sample.

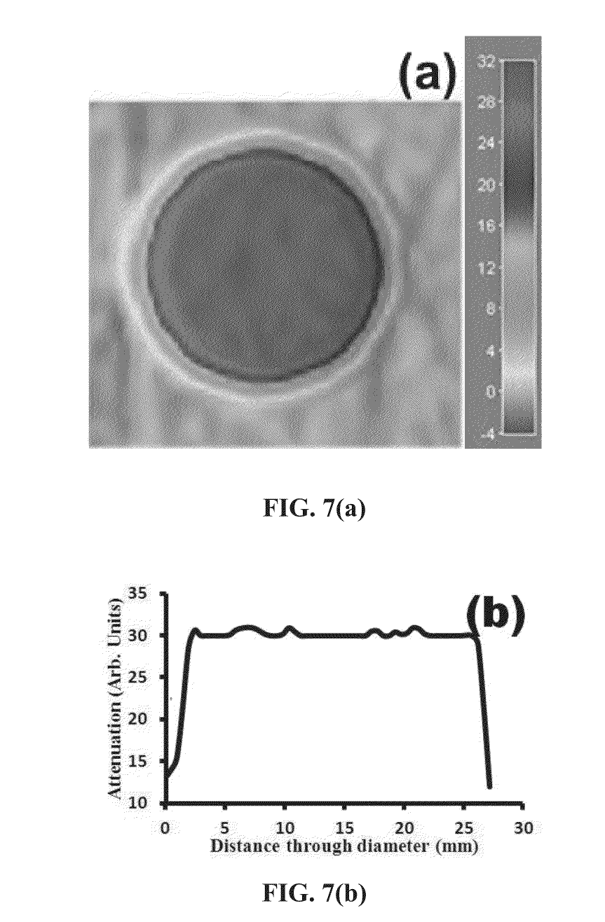

The correction algorithm described above for certain embodiments of the present invention is applied to all of the projection data as a function of sample rotation from the homogenous plexiglass rod and the results are shown in FIG. 7. Comparing the reconstructed image with (FIG. 7(a)) and without (FIG. 1(b)) correction clearly shows that the anomolously large attenuation near the boundaries of the rod have been removed in the reconstructed image. FIG. 7(b) shows a profile of the reconstructed localized attenuation coefficient taken along a diameter through FIG. 7(a). Note that the localized attenuation coefficent is nearly constant across the sample as would be expected for a homogeneous sample.

THz rays which pass near the boundary of the object are subjected to severe beam steering. For a homogenous material for which Eq. (1) is a good representation of the true sample attenuation, there is not much error in the reconstructed image by using Eq. (1) to match the projection array data from the physical edge of the sample to the regions for the measured attenuation is accurately measured. However, if a structural feature were present in the peripheral region of the sample, the measured attenuation—since it is dominated by the blocking of the THz beam by the sample's edge—would not be representative of the structure feature. In essence, the measured THz attenuation near the periphery of the sample is “blind” to the presence of any structural features which might cause either an increase or decrease in the measured attenuation. For the blind regions, no real information about the internal structure of the object can be obtained.

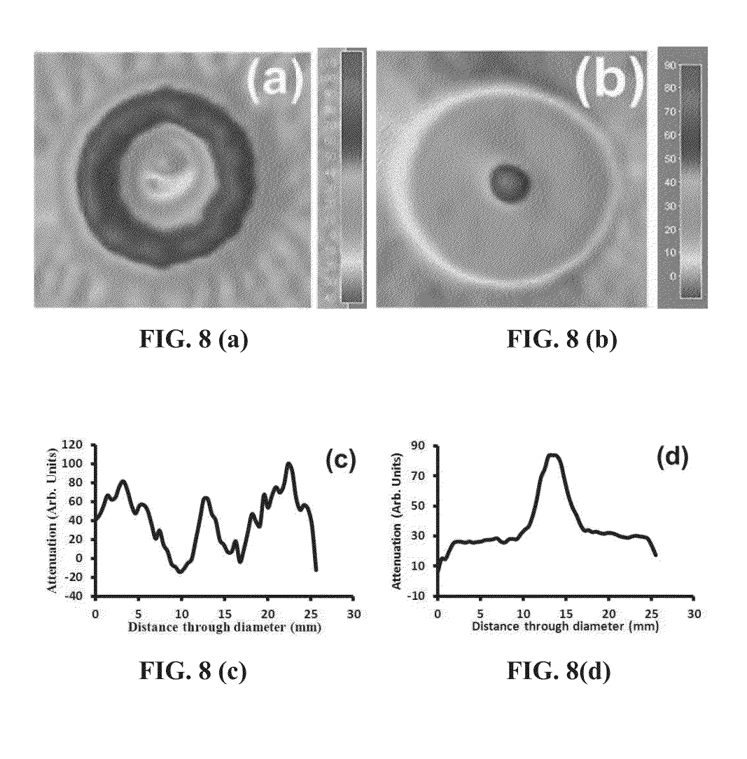

In order to test the sensitivity of the algorithms of certain embodiments described herein for the presence of defects near the periphery of a sample, the algorithms were applied to three plastic rods with defects. The reconstructed images of the rod with a 1 mm hole near the center are shown both without (FIG. 8(a)) and with application of the correction algorithm (FIG. 8(b)). The reconstructed local absorbance along slices through the images are shown in FIGS. 8(c) and 8(d). The reconstructed hole appears as a spot of large localized attenuation because the hole itself acts as a beam steering obstruction which deviates the THz rays resulting in an increased of attenuation. The reconstructed hole size appears larger than the actual size of the hole which is attributed to the finite size a₀=2 mm of the THz beam.

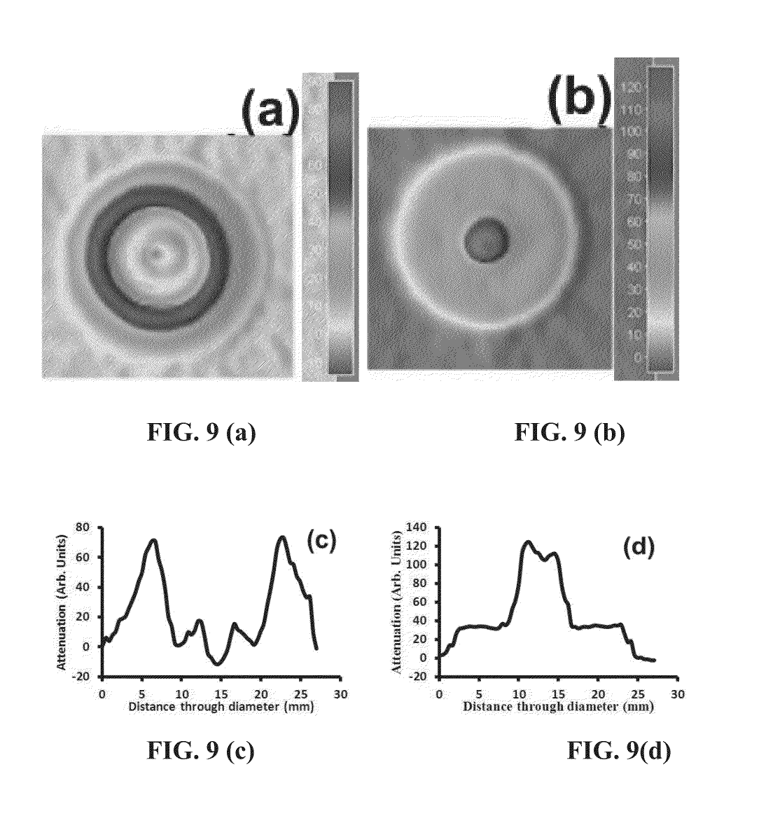

FIGS. 9(a)-(d) show the 2D THz-CT reconstructed images and plot profiles of a plastic rod with a 5 mm hole near the center region. Without correcting for the boundary effects, the presence of the 5 mm hole in the reconstructed image (FIG. 9(a)) is not as prominent as when the correction algorithms are applied. After correction, both the shape and approximate size of the defect are reconstructed in FIG. 9(b) and FIG. 9(d). With reference to FIGS. 8(a)-(d) and FIGS. 9(a)-(d), it can be seen the hole in the rod steers the THz from its nominal direction leading to an increase in measured attenuation. Clearly, the increase in attenuation due to the presence of the hole is much more prominent when the boundary effects are eliminated using the correction algorithms. These results suggest that the application of the correction algorithms should work reasonably well if any defects are in the central region of the plastic rods.

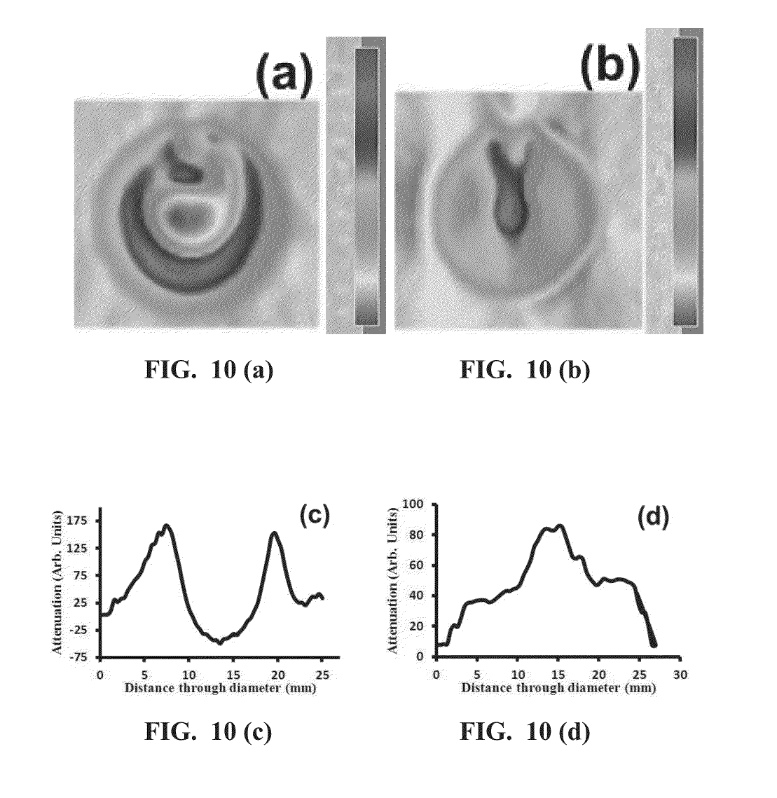

However, when the defect in the rods is located in the “blind” region near the physical boundaries of the rod, discerning the hole near the edge becomes difficult. With reference to FIGS. 10(a)-(d), 2D reconstructed images and plot profiles of a plastic rod having a 5 mm diameter hole near to the edge of the rod are shown. In comparing FIG. 10(a) with FIG. 1(b), it is clear that the distortion of the uncorrected reconstructed image near the top edge of the rod corresponds to a defect in the material, but the shape of the defect is unrecognizable. When the correction algorithm is applied, the resulting reconstructed image does show the presence of a defect in the material (comparing FIG. 10(b) with FIG. 8(a)), but neither the shape nor the exact location of the defect is accurately determined. The source of the error is understood to result from incorrect projection data as a function of sample rotation. For a certain range of rotation angles, the hole near the periphery of the sample is in a “blind” spot. For other angles, the probing THz radiation will interact with the hole and manifest the presence of the hole through an increase in the measured attenuation. The presence of the hole in attenuation projection data for some but not all of the rotation angles leads to errors in the reconstructed image.

In one embodiment of the present invention correction algorithms are applied to attenuation projection data from natural cork wine stoppers. For this material, the presence of lenticel structures as well as internal cracks and voids are characterized by an increase in the measured THz attenuation. See, Y. Hor, J. F. Federici, and R. L. Wample, Applied Optics, 2008. 47: p. 72-78, incorporated herein by reference in its entirety. Lenticels are naturally occurring cell structures which enable the exchange of gases between the atmosphere and the interior of plant tissues. While an accurate reconstruction of an object with arbitrary shape and composition by THz CT is problematic due to refraction artifacts, THz CT can still be an effective NDE tool for corks since the size and shape of cork stoppers are standardized. Moreover, the relatively low refractive index (approximately 1.1) of natural cork reduces both the refractive and beam steering effects and the volume of “blind” volumes within the cork relative to the Plexiglas samples discussed above. Removing the boundary artifacts enables a more detailed reconstruction of each sample's internal structure which is so critical to the functionary of the stoppers with regards to their gas and liquid diffusion properties.

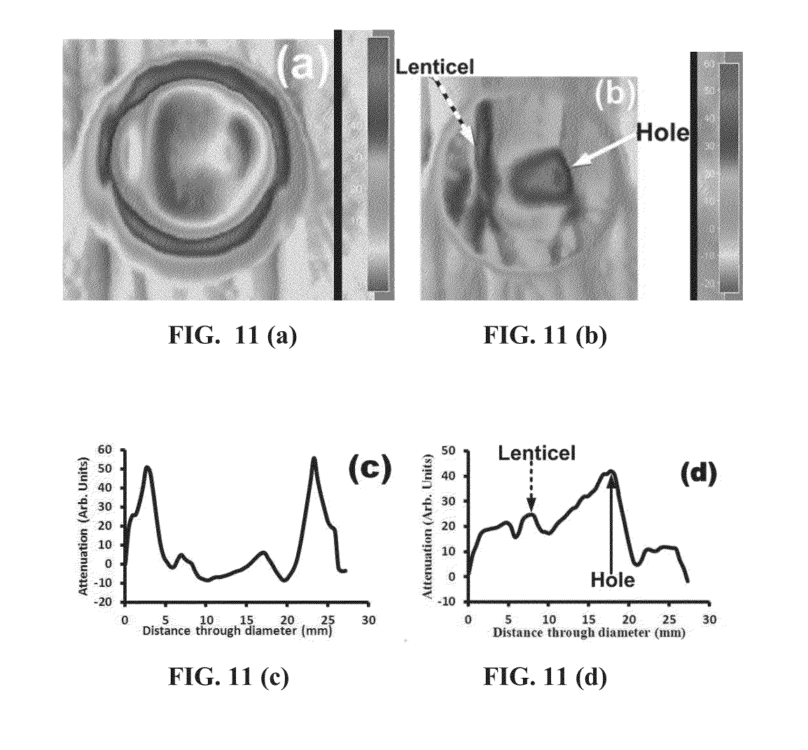

Correction algorithms of certain embodiments of the present invention were tested on a cylindrically shaped natural cork stopper. Cork samples were tested with a ˜3 mm diameter hole which was bored into the sample by an insect. With reference to FIGS. 11(a) and (b), the round spot near the solid arrow is an insect hole in the cork while the dashed arrow indicates a lenticular channel in the cork structure. This insect hole is analogous to the holes which were drilled into the plastic rod samples. FIG. 11(a) shows the 2D reconstructed THz-CT image of the cork sample. Since cork has a lower refractive index than plastic, the bending and steering of the THz beam is much less inside the cork than inside the plastic. Without application of the correction algorithms of certain embodiments of the present invention, FIG. 11(a) shows a prominent boundary surrounding the cork. However, when the correction algorithm is applied (FIG. 11(b)), the boundary is removed and more of the cork's internal structure is revealed. The arrows in FIG. 11(b) illustrate the location of the insect hole and lenticel channel in the cork which is not visible in FIG. 11(a). Plots of the reconstructed attenuation through the diameter of the cork (FIGS. 11(c) and (d)) illustrate that the correction algorithms remove the boundary artifact and enable visualization of the sample's internal structure.

The correction algorithm and procedure applied to cork may be described as follows:

In one embodiment of the present invention, a cylindrical cork is used. The dimensions of the cork may be about 45 mm in length and a diameter of about 28 mm. Cork having other dimensions may be used as long as the cork sample to be tested is roughly cylindrical in shape.

The cork sample is scanned horizontally at 1 mm intervals and vertically at 5 mm intervals. The minimum scanning limit is 10 μm minimum and there is no maximum scanning limit. In further embodiments of the present invention horizontal and/or vertical intervals can be in the range of 10 μm to a maximum of the horizontal and vertical dimension of the cork used.

For each projection slice, Eq. (10) is used to determine the location of the left and right edges of the cork relative to the center of rotation of the sample.

Based on the maximum detectable attenuation (typically ˜4.6), the range of l_{R} values for which the measured attenuation is not instrument limited is determined. This limit of the maximum detectable attenuation is instrument specific and it could be any arbitrary number, more or less than 4.6 and it could vary depending upon the instrument used. The correction terms of Eq. (7) are applied to that range of l_{R} parameters.

The ideal form for the attenuation of Eq. (1) is used to fill in projection array data as a function of l_{R} in the “blind” region from the cork's edges to the boundary of the valid corrected data. The procedure is repeated for each projection slice for all rotation angles of the cork.

Although the systems and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention. All references cited and/or listed herein are incorporated by reference herein in their entireties.

### REFERENCES

1. B. B. Hu and M. C. Nuss, Optics Letters. 20(16): p. 1716-1718 (1995).
2. D. Mittleman, IEEE Journal of selected optics in quantum electronics, 2(3): p. 679-692 (1996).
3. S. Wang and X-C Zhang, Journal of Physics D, 37(4): p. R1-R36 (2004).
4. D. Mittleman, S. Hunsche, L. Boivin, and M. C. Nuss, Optics Letters, 22(12): p. 904-906 (1997).
5. A. Brahm, M. Bauer, T. Hoyer, H. Quast, T. Loeffler, S. Riehemann, G. Notni, and A. Tunnermann, Infrared, Millimeter and Terahertz Waves (IRMMW-THz), 2011 36th International Conference: (2011).
6. T. Buma and T. B. Norris, Applied Physics Letters, 84(12): p. 2196-2198 (2004).
7. Wenfeng Sun, Xinke Wanl, Ye Cui, and, and Yan Zhang, The European Conference on Lasers and Electro-Optics, Munich, Germany: (2011).
8. Li. Qi, Li. Yun-Da, D. Sheng-Hui, and W. Qi, Journal of Infrared, Millimeter, and Terahertz Waves, 33(5): p. 548-558 (2012).
9. A. Wei-Min-Lee, K. Tsung-Yu, D. Burghoff, Q. Hu, and J-.Reno, Opt. Lett., 37(2): p. 217-219(2012).
10. B. Ferguson, S. Wang, D. Gray, D. Abbot, and X-C. Zhang, Optics Letters, 27(15): p. 1312-1314 (2002).
11. M. Bessou, B. Chassagne, J-P. Caumes, C. Pradere, P. Maire, M. Tondusson, and E. Abraham, Appl. Opt., 51(28): p. 6738-6744 (2012).
12. D. J. Roth, S. B. Reyes-Rodriguez, D. A. Zimdars, R. W. Rauser, and W. W. Ussery, 69(9): Materials Evaluation, p. 1090-1098 (2011).
13. B. Recur, A. Younus, S. Salort, P. Mounai, B. Chassagne, P. Desbarats, J-P. Caumes, and E. Abraham, Optics Express, 19(6): p. 5105-5117 (2011).
14. Y. Hor, J. F. Federici, and R. L. Wample, Applied Optics, 2008. 47: p. 72-78.
15. S. Mukherjee and J. F. Federici, IRMMW-THz 2011—36th International Conference on Infrared, Millimeter, and Terahertz Waves, Houston, Tex., USA: p. 85 (2011).
16. A. J. Teti, D. E. Rodriguez, J. F. Federici, and C. Brisson, J. Infrared Milli. Terahz. Waves, 32: p. 513-527 (2011).
17. J. F. Federici, IEEE Transactions on Terahertz Science and Technology, 33(2): p. 97-126 (2012).
18. B. Ferguson, S. Wang, D. Gray, D. Abbott, and X-C. Zhang, Physics in Medicine and Biology, 47(21): p. 3735-3742 (2002).
19. X-C. Zhang, Philosophical Transactions of The Royal Society A, 362: p. 283-299 (2003).
20. W. L. Chan, J. Deibel, and D. M. Mittleman, Reports on Progress in Physics, 70(8): p. 1325-1379 (2007).
21. K. Hyeongmun, K. Kyung-Won, S. Joo-Hiuk, and H. Joon-Koo, Optical Society of America (2012).
22. S. A. Nishina, K. A. Takeuchi, M. A. Shinohara, M. A. Imamura, M. B. Shibata, Y. B. Hashimoto, and F. B. Watanabe, SAE International Journal of Fuels and Lubricants, 5(1): p. 343-351 (2012).
23. B. Recur, J. P. Guillet, I. Manek-flonninger, J. C. Delagnes, W. Benharbone, P. Desbarats, J. P. Domenger, L. Canioni, and P. Mounaix, Optics Express, 20(6): p. 5817-5829 (2012).
24. E. Abraham, A. Younus, C. Aguerre, P. Desbarats, and P. Mounaix, Optics Communications, 283(10): p. 2050-2055 (2010).
25. Eugene Hecht, Optics, 4^{th} edn, (Addison Wesley, 2001).
26. E. D. Hirleman, and, and W. H. Stevenson, Applied Optics, 17(21): (1978).
27. H. Pereira, Cork: Biology, Production and Uses, (New York: Elsevier. 2007).

What is claimed is:

1. A method of reducing boundary effect in a THz-CT image comprising correcting steering of the THz beam and/or Fresnel reflection prior to reconstruction of the THz-CT image, the method comprising tomographically scanning an object at selected rotational positions to obtain a plurality of projection slices, and prior to reconstructing the THz-CT image, applying to each of the plurality of projection slices an algorithm
$${A}_{edge} ( {l}_{R} ) = - {log}_{e} [ \frac{1}{2} + \frac{1}{2} erf ( \frac{\sqrt{2} R}{{a}_{0}} ( {l}_{R} - 1 ) ) ]$$
to determine a location of left and right edges of the object relative to the center of rotation of the object, determining a range of l_{R} values for which the measured attenuation is not instrument limited based on a maximum detectable attenuation, applying an algorithm comprising the correction terms αLᵣ=−ln(T(l_{R}))+ln(Tₛₜ(l_{R}))+ln(1−Rₚₐ)±ln(1−Rₐₚ) to the range of l_{R} values, wherein αLᵣ represents the corrected attenuation projection array data, −ln(T(l_{R})) is an experimentally measured attenuation, ln(Tₛₜ(l_{R})) is the correction for beam steering, and ln(1−Rₚₐ) and ln(1−Rₐₚ) account for Fresnel reflection loss at the incident and exiting air-object interfaces.

2. The method according to claim 1 wherein the object is cylindrical.

3. The method according to claim 1 comprising applying an algorithm Aₜₕ(l_{R})=α₀L(l_{R})=α₀2R\sqrt{1−l_{R}²} where l_{R}=l/R and R is the radius of the object, to fill in projection array data as a function of l_{R} in a blind region from the object's edges to a boundary of corrected data.

4. The method according to claim 1 wherein the object is plastic.

5. The method according to claim 1 wherein the object is natural cork.

6. The method according to claim 5 wherein the natural cork is scanned horizontally at 1 mm intervals and vertically at 5 mm intervals.

7. The method according to claim 5 comprising applying an algorithm Aₜₕ(l_{R})=α₀L(l_{R})=α₀2R\sqrt{1−l_{R}²} where l_{R}=l/R and R is the radius of the object, to fill in projection array data as a function of l_{R} in a blind region from the cork's edges to a boundary of corrected data.

8. The method according to claim 1 comprising reconstructing the THz-CT image using a Radon transformation.

9. A non-transitory, computer readable storage medium containing a computer program, which when executed by a computer processor causes the computer processor to perform actions, the actions comprising:
correcting steering of a THz beam and/or Fresnel reflection prior to reconstruction of a THz-CT image, comprising applying to each of a plurality of projection slices obtained by tomographically scanning an object at selected rotational positions an algorithm
$${A}_{edge} ( {l}_{R} ) = - {log}_{e} [ \frac{1}{2} + \frac{1}{2} erf ( \frac{\sqrt{2} R}{{a}_{0}} ( {l}_{R} - 1 ) ) ]$$
to determine a location of left and right edges of the object relative to the center of rotation of the object, determining a range of l_{R} values for which the measured attenuation is not instrument limited based on a maximum detectable attenuation, applying an algorithm comprising the correction terms αLᵣ=−ln(T(l_{R}))+ln(Tₛₜ(l_{R}))+In(1−Rₚₐ)±ln(1−Rₐₚ) to the range of l_{R} values, wherein αLᵣ represents the corrected attenuation projection array data, −ln(T(l_{R})) is an experimentally measured attenuation, ln(Tₛₜ(l_{R})) is the correction for beam steering, and ln(1−Rₚₐ) and ln(1−Rₐₚ) account for Fresnel reflection loss at the incident and exiting air-object interfaces.

10. The non-transitory, computer readable storage medium of claim 9 wherein the object is cylindrical.

11. The non-transitory, computer readable storage medium according to claim 9 comprising applying an algorithm Aₜₕ(l_{R})=α₀L(l_{R})=α₀2R\sqrt{1−l_{R}²} where l_{R}=l/R and R is the radius of the object, to fill in projection array data as a function of l_{R} in a blind region from the object's edges to a boundary of corrected data.

12. The non-transitory, computer readable storage medium according to claim 9 wherein the object is natural cork.

13. The non-transitory, computer readable storage medium according to claim 12 comprising applying an algorithm Aₜₕ(l_{R})=α₀L(l_{R})=α₀2R\sqrt{1−l_{R}²} where l_{R}=l/R and R is the radius of the object, to fill in projection array data as a function of l_{R} in a blind region from the cork's edges to a boundary of corrected data.

14. An apparatus, including a processor operating to perform actions in response to executing computer program instructions, the actions comprising:
correcting steering of a THz beam and/or Fresnel reflection prior to reconstruction of a THz-CT image, comprising applying to each of a plurality of projection slices obtained by tomographically scanning an object at selected rotational positions an algorithm
$${A}_{edge} ( {l}_{R} ) = - {log}_{e} [ \frac{1}{2} + \frac{1}{2} erf ( \frac{\sqrt{2} R}{{a}_{0}} ( {l}_{R} - 1 ) ) ]$$
to determine a location of left and right edges of the object relative to the center of rotation of the object, determining a range of l_{R} values for which the measured attenuation is not instrument limited based on a maximum detectable attenuation, applying an algorithm comprising the correction terms αLᵣ=−ln(T(l_{R}))+ln(Tₛₜ(l_{R}))+In(1−Rₚₐ)+ln(1−Rₐₚ) to the range of l_{R} values, wherein αLᵣ represents the corrected attenuation projection array data, −ln(T(l_{R})) is an experimentally measured attenuation, ln(Tₛₜ(l_{R})) is the correction for beam steering, and ln(1−Rₚₐ) and ln(1−Rₐₚ) account for Fresnel reflection loss at the incident and exiting air-object interfaces.

15. The apparatus of claim 14 wherein the object is cylindrical.

16. The apparatus of claim 14 comprising applying an algorithm Aₜₕ(l_{R})=α₀L(l_{R})=α₀2R\sqrt{1−l_{R}²} where l_{R}=l/R and R is the radius of the object, to fill in projection array data as a function of l_{R} in a blind region from the object's edges to a boundary of corrected data.

17. The apparatus of claim 14 wherein the object is natural cork.

18. The apparatus of claim 14 comprising applying an algorithm Aₜₕ(l_{R})=α₀L(l_{R})=α₀2R\sqrt{1−l_{R}²} where l_{R}=l/R and R is the radius of the object, to fill in projection array data as a function of l_{R} in a blind region from the cork's edges to a boundary of corrected data.

19. A method of reducing boundary effect in a THz-CT image comprising correcting steering of the THz beam and/or Fresnel reflection prior to reconstruction of the THz-CT image, the method comprising tomographically scanning an object at selected rotational positions to obtain a plurality of projection slices, and prior to reconstructing the THz-CT image, applying to each of the plurality of projection slices an algorithm to determine a location of left and right edges of the object relative to the center of rotation of the object, determining a range of values for which the measured attenuation is not instrument limited based on a maximum detectable attenuation, and applying a further algorithm representing the corrected attenuation projection array data, the algorithm comprising an experimentally measured attenuation, correction for beam steering, and accounting for Fresnel reflection loss at the incident and exiting air-object interfaces.

20. The method according to claim 19 comprising reconstructing the THz-CT image using a Radon transformation.
